(19) United States
(12) Patent Application Publication
(10) Pub. No.: US 2026/0266639 A1
(43) Pub. Date: Sep. 10, 2026

(54) MICRO-ELECTRO-MECHANICAL SYSTEMS ON FIBER SENSORS FOR FLUID CHARACTERIZATION, FLOW, AND DENSITY MEASUREMENT

(71) Applicant: Saudi Arabian Oil Company, Dhahran (SA)

(72) Inventors: Meshal Alluqmani, Dhahran (SA); Muhammad Arsalan, Dhahran (SA)

(21) Appl. No.: 19/072,670

(22) Filed: Mar. 6, 2025

(51) Int. Cl. G01F 1/66; G01F 1/36; G01F 1/667; G01N 33/28

(52) U.S. Cl. CPC G01F 1/662; G01F 1/36; G01F 1/667; G01N 33/2841; G01N 33/2847

(57) ABSTRACT

Technologies for multiphase flow measurement using micro-electro-mechanical sensing are described. An electromagnetic signal and an acoustic signal generated by reflections of a multiphase mixture flowing through a pipe are received. A differential pressure of the multiphase mixture is received. Flow rates of the multiphase mixture are received from a microwave-based sensor system. A water content of the multiphase mixture flowing through the pipe is determined by processing the electromagnetic signal. A flow velocity and a water-cut of the multiphase mixture flowing through the pipe is determined by processing the acoustic signal. A mass velocity of the multiphase mixture flowing through the pipe is determined by processing the differential pressure of the multiphase mixture. A composition of the multiphase mixture of the pipe in the industrial flow loop is estimated based on the water content, the flow velocity, and the mass velocity of the multiphase mixture.

### TECHNICAL FIELD

This disclosure relates to multiphase flows in the oil and gas industry and, more specifically, to multiphase flow measurement using micro-electro-mechanical sensing.

### BACKGROUND

Some multiphase flow meters utilize radioactive material-based technology: Radioactive based measurements of phase volume fractions in multiphase flows employ radiation, such as neutrons, gamma rays, or x-rays, that is attenuated by the flow. Measuring radiation attenuation through the flow provides information on local density or phase fraction. Handling radioactive materials poses health and safety risks associated with the use of radioactive materials. The radioactive multiphase flow meters raise the possibility of unwanted exposure to radiation if the meter is damaged or mishandled.

### SUMMARY

Implementations of the present disclosure are directed to multiphase flows in the oil and gas industry. More particularly, implementations of the present disclosure are directed to multiphase flow measurement using micro-electro-mechanical sensing.

In some implementations, a method includes: receiving, by one or more processors from a light receiver, an electromagnetic signal generated by an electromagnetic wave reflected by a multiphase mixture flowing through a pipe, receiving, by the one or more processors from a transducer, an acoustic signal reflected by the multiphase mixture flowing through the pipe, receiving, by the one or more processors from a differential pressure sensor, a differential pressure of the multiphase mixture flowing through the pipe, determining, by the one or more processors, a water content of the multiphase mixture flowing through the pipe from the electromagnetic signal, determining, by the one or more processors, a flow velocity and a water-cut of the multiphase mixture flowing through the pipe from the acoustic signal, determining, by the one or more processors, a mass velocity of the multiphase mixture flowing through the pipe from the differential pressure of the multiphase mixture, estimating, by the one or more processors, a composition of the multiphase mixture by determining gas, water, and oil volumetric flow rates based on the water content, the flow velocity, and the mass velocity of the multiphase mixture flowing through the pipe, and triggering, by the one or more processors, an adjustment of a setting of an industrial equipment based on the composition of the multiphase mixture.

The foregoing and other implementations can optionally include one or more of the following features, alone or in combination. In particular, implementations can include all the following features:

In a first aspect, combinable with any of the previous aspects, wherein the light receiver is placed inside an opening of the pipe being exposed to the multiphase mixture flowing through the pipe. In another aspect, combinable with any of the previous aspects, the light receiver includes an infrared sensor. In another aspect, combinable with any of the previous aspects, the transducer includes an optoacoustic transducer and an acoustic receiver. In another aspect, combinable with any of the previous aspects, the differential pressure sensor includes a flow restrictor producing a pressure change in the multiphase mixture flowing through the pipe. In another aspect, combinable with any of the previous aspects, determining, by the one or more processors, the water content includes determining, by the one or more processors, a gas volume fraction from a mixture density. In another aspect, combinable with any of the previous aspects, the computer-implemented method further includes correlating the gas volume fraction to the water-cut of the multiphase mixture flowing through the pipe. In another aspect, combinable with any of the previous aspects, the light receiver, the transducer and the differential pressure sensor are included in a housing, at least a portion of the housing being inserted within the pipe through a wall gap.

Other implementations of the aspect include corresponding systems, apparatus, and computer programs, configured to perform the actions of the methods, encoded on computer storage devices.

The present disclosure also provides a computer-readable storage medium coupled to one or more processors and having instructions stored thereon which, when executed by the one or more processors, cause the one or more processors to perform operations in accordance with implementations of the methods provided herein.

The present disclosure further provides a system for implementing the methods provided herein. The system includes one or more processors, and a computer-readable storage medium coupled to the one or more processors having instructions stored thereon which, when executed by the one or more processors, cause the one or more processors to perform operations in accordance with implementations of the methods provided herein.

It is appreciated that methods in accordance with the present disclosure can include any combination of the aspects and features described herein. That is, methods in accordance with the present disclosure are not limited to the combinations of aspects and features specifically described herein, but also include any combination of the aspects and features provided.

Implementations described in the present disclosure, provide multiple technical advantages. For example, the multiphase flow measurement using safe (non-radioactive) multi-sensing technology. The described technology relates to methods and systems for measuring volumetric multiphase flows in the oil and gas industry by combining data acquired by an infrared water cut meter with optoacoustic transducers and differential pressure sensors. Differential pressure data is processed to determine the mass velocity. The wave response detected by the infrared transducer is used to measure the water-cut. The travel time of the transmitted acoustics signal measured from the optoacoustic transducers corresponds to the speed of sound that can be correlated to the mixture density. Another advantage of the described technology is that the mass velocity, the water-cut, and the gas volumetric flow rate are integrated to derive the gas, water, and oil volumetric flow rates. Another advantage of the described technology is that the described multi-sensing technology presents a promising solution for accurate and efficient multiphase flow measurement in industrial settings that facilitates optimization of industrial machine and device operations for safe continuation of industrial operations and optimization of hydrocarbon management within industrial plants.

The details of one or more implementations of the subject matter of the specification are set forth in the accompanying drawings and the description below. Other features, aspects, and advantages of the subject matter can become apparent from the description, the drawings, and the claims.

### DESCRIPTION OF DRAWINGS

The accompanying drawings, which are incorporated in and constitute a part of this specification, show particular aspects of the subject matter disclosed herein and, together with the description, help explain some of the principles associated with the disclosed implementations. In the drawings,

FIG. 1 is a is a block diagram illustrating an example system for multiphase flow measurement that can be used to execute implementations of the present disclosure;

FIG. 2A illustrates an example data collection system that can be used to execute implementations of the present disclosure;

FIG. 2B illustrates another example data collection system that can be used to execute implementations of the present disclosure;

FIG. 2C illustrates another example data collection system that can be used to execute implementations of the present disclosure;

FIG. 2D illustrates another example data collection system that can be used to execute implementations of the present disclosure;

FIG. 2E illustrates another example data collection system that can be used to execute implementations of the present disclosure;

FIG. 2F illustrates another example data collection system that can be used to execute implementations of the present disclosure;

FIG. 3 illustrates an example process that can be used to execute implementations of the present disclosure;

FIG. 4 depicts a block diagram illustrating a computing system, in accordance with some example implementations; and

FIG. 5 illustrates hydrocarbon production operations, in accordance with some example implementations.

When practical, like labels are used to refer to same or similar items in the drawings.

### DETAILED DESCRIPTION

Implementations of the present disclosure are directed to multiphase flows in the oil and gas industry. More particularly, implementations of the present disclosure are directed to multiphase flow measurement using micro-electro-mechanical sensing. An electromagnetic signal generated by an electromagnetic wave reflected by a multiphase mixture flowing through a pipe is received. An acoustic signal reflected by the multiphase mixture flowing through the pipe is received. A differential pressure, detected by a differential pressure sensor inserted in the multiphase mixture flowing through the pipe is received. The multiphase mixture includes oil, water, and gas, flow rates of the multiphase mixture are received from a microwave-based sensor system. A water content of the multiphase mixture flowing through the pipe is determined by processing the electromagnetic signal. A flow velocity and a water-cut of the multiphase mixture flowing through the pipe is determined by processing the acoustic signal. A mass velocity of the multiphase mixture flowing through the pipe is determined by processing the differential pressure of the multiphase mixture. A composition of the multiphase mixture of the pipe in the industrial flow loop is estimated based on the water content, the flow velocity, and the mass velocity of the multiphase mixture. An adjustment of a setting of an industrial equipment is triggered based on the composition of the multiphase mixture.

Some radioactive and non-radioactive based multiphase flow meters have been implemented to characterize volume fractions of a complex and variable blend of oil, water, and gases, commonly referred to as multiphase mixture. The radioactive based multiphase flow meters use waves that can penetrate the dielectric pipe materials, exposing personnel to health risks. Non-radioactive that characterize the fluid composition include infrared, microwave, and capacitance technologies. Infrared technology is limited to situations with a high water cut with an insignificant amount of gas. Water, being a polar molecule, has a higher dielectric constant than oil and gases, while gases have insignificant dielectric loss as compared to liquids (oil and water). Some microwave resonance-based two-phase sensors rely on the change in the resonance frequency as well as the quality factor to measure the water content in oil (0-100%). Variations in temperature and salinity levels can affect the accuracy of microwave measurements. Microwave technology can also significantly depend on the proportion of the full pipe profile that was captured. Capacitance technology is unable to perform effectively in high water cut situations when the fluid is in a continuous water phase. Conductance technology can be used in conjunction with capacitive technology to enhance measurements in the water-continues zone (conductive mixture). The influence of the fluid properties, salinity, temperature, and pressure can limit the ability to precisely define a transition zone between two stages of a multiphase mixture, which generally involves a non-linear inter-phase progression.

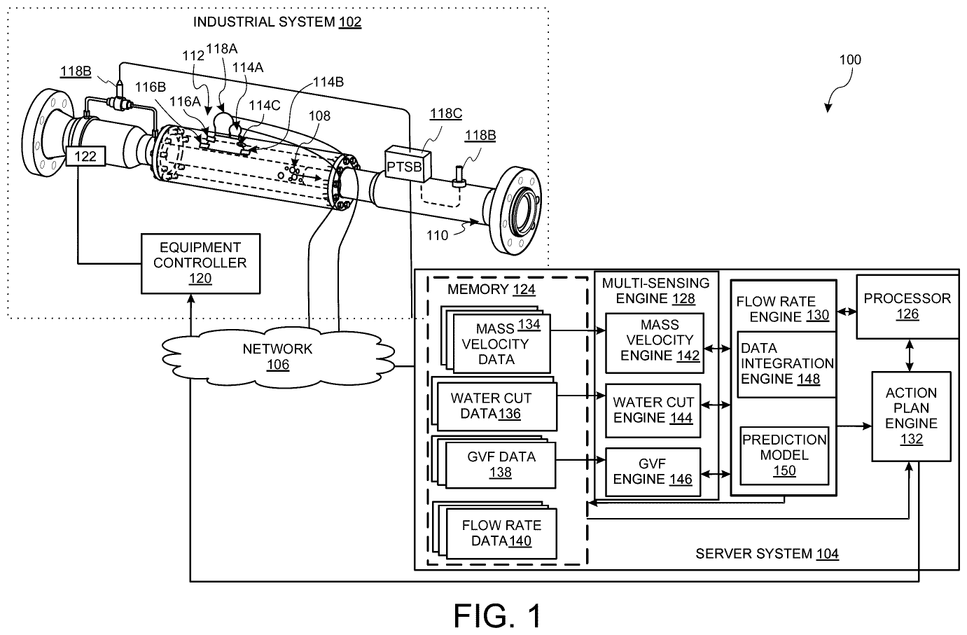

Addressing the challenges of traditional radioactive and non-radioactive based sensing of multiphase flows in the oil and gas industry, the multi-sensing-based multiphase flow measurement described in the present disclosure provides an accurate and safe multiphase mixture characterization. The described technology relates to methods and systems for measuring volumetric multiphase flows in the oil and gas industry by combining data acquired by an infrared water cut meter with optoacoustic transducers and differential pressure sensors. Differential pressure data is processed to determine the mass velocity. The wave response detected by the infrared transducer is used to measure the water-cut. The travel time of the transmitted acoustics signal measured from the optoacoustic transducers corresponds to the speed of sound that can be correlated to the mixture density. Another advantage of the described technology is that the mass velocity, the water-cut, and the gas volumetric flow rate are integrated to accurately derive the gas, water, and oil volumetric flow rates. Another advantage of the described technology is that the described multi-sensing technology presents a promising solution for accurate and efficient multiphase flow measurement in industrial settings that facilitates optimization of industrial machine and device operations for safe continuation of industrial operations and optimization of hydrocarbon management within industrial plants. Other advantages of the multiphase flow measurement using multi-sensing techniques are described with reference to FIGS. 1-5.

FIG. 1 is a block diagram illustrating an example system 100 for multiphase flow measurement that can be used to execute implementations of the present disclosure. The example system 100 includes or is communicably coupled with an industrial system 102, a server system 104, and a network 106. Although shown separately, in some implementations, functionality of two or more systems or components of the example system 100 can be provided by multiple computing devices, a computing device connected to a computing system or a server. In some implementations, the functionality of one illustrated system, computing device, or component can be provided by multiple systems, servers, or components, respectively.

In general, the industrial system 102 monitors and controls a flow of a multiphase mixture 108 flowing through an industrial pipe 110. The multiphase flow measurement of a multiphase mixture 108 can be performed using a sensor system 112. The sensor system 112 performs time-based flow measurements for the determination of water-fraction content in the presence of three-phases (oil, water, and gas), and further allows for the prediction of flow patterns of the fluid passing through the industrial pipe 110.

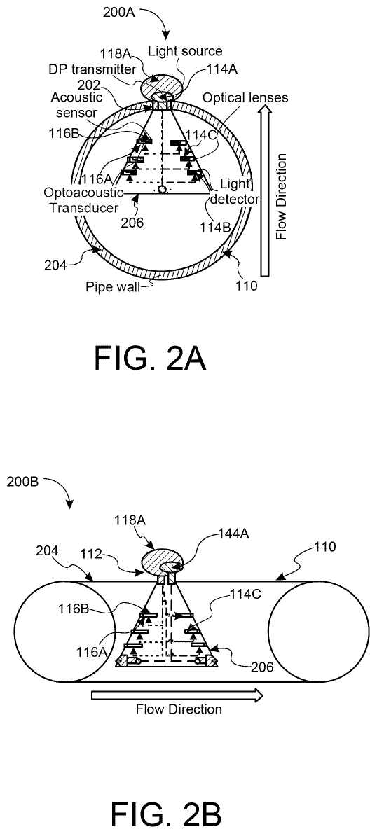

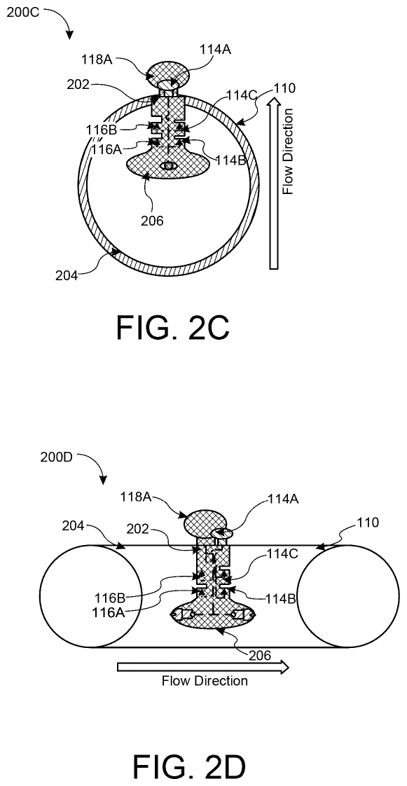

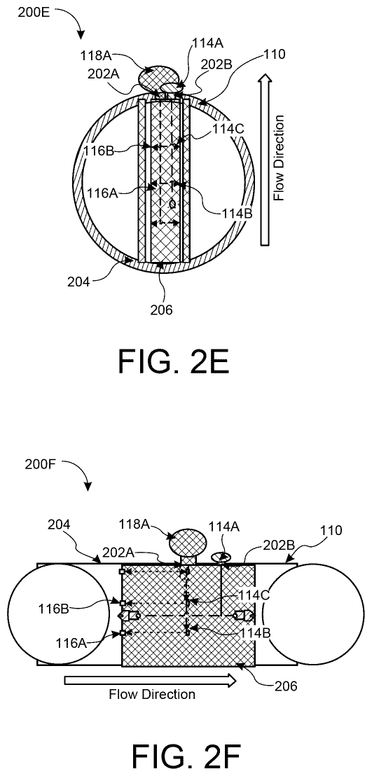

The sensor system 112 includes a light source 114A, a light sensor 114B, one or more optical lenses 114C, an optoacoustic transducer 116A, an acoustics receiver 116B, and a differential pressure sensor 118A. In some implementations, the sensor system 112 includes a temperature transducer 118B and a pressure/temperature sensor box 118C that can generate additional flow measurement data indicative of the flow of the multiphase mixture 108 flowing through an industrial pipe 110. The light sensor 114B can collect light that was generated by the light source 114A, reflected by the multiphase mixture, and focused by the one or more optical lenses 114C. The acoustics receiver 116B can collect acoustic signals generated by the optoacoustic (infrared) transducer 116A and reflected by the multiphase mixture. The differential pressure sensor 118A can act as a flow restrictor and can measure the multiphase mixture flow velocity for a dominant phase (a phase defining the greatest percentage of a composition of the multiphase mixture). The recording is repeated at set intervals (e.g., every 100 milliseconds or “ms”) so that the dynamics of the change in the flow of the multiphase mixture 108 can be captured. Further details about the sensor system 112 are provided with respect to FIGS. 2A-2F.

The industrial system 102 includes an equipment controller 120 configured to activate a flow controller 122 that controls the flow of the multiphase mixture 108 flowing through an industrial pipe 110. For example, the equipment controller 120 can trigger a modification of a setting of the flow controller 122 for adjusting pressure, flow rate, and/or volume of the multiphase mixture 108 flowing through an industrial pipe 110. The flow controller 122 can include a valve and/or a pump that can regulate any of the pressure, flow rate, and/or volume of the multiphase mixture 108 flowing through an industrial pipe 110.

The server system 104 is intended to represent various forms of servers including, but not limited to a web server, an application server, a proxy server, a network server, and/or a server pool. In general, the server system 104 manages multiphase flow measurement. In accordance with implementations of the present disclosure, and as noted above, the server system 104 can host a solution environment that can be a cloud environment providing software applications, systems, and services that can be consumed by customers as a service. In some implementations, the server system 104 can support multiphase flow measurement of different multiphase mixtures through different pipe types, as well as services of different types that are integrated in customer integration scenarios and support execution of defined processes.

For example, the server system 104 includes a memory 124, a processor 126, a multi-sensing engine 128, a flow rate engine 130, and an action plan engine 132. The memory 124 can include any type of memory or database module and can take the form of volatile and/or non-volatile memory including, without limitation, magnetic media, optical media, random access memory (RAM), read-only memory (ROM), removable media, or any other suitable local or remote memory component. The memory 124 can store various objects or data, including caches, classes, frameworks, applications, backup data, objects, jobs, web pages, web page templates, database tables, database queries, repositories storing safety data and/or dynamic information, and any other appropriate information including any parameters, variables, algorithms, instructions, rules, constraints, or references thereto associated with the purposes of the industrial system 102 and the server system 104, respectively.

The memory 124 can store raw data (e.g., data received from the industrial system 102, such as live monitoring data) and processed data (e.g., outputs of the multi-sensing engine 128). For example, the memory 124 can store including mass velocity data 134, water cut data 136, gas volume fractions (GVF) data 138, and flow rate data 140 determined by the multi-sensing engine 128 including a mass velocity engine 142, a water cut engine 144, and a GVF engine 146. The mass velocity data 134 can be determined by the mass velocity engine 142 that processes data measured by the differential pressure transducer 118A and received from the industrial system 102. The water cut data 136 can be determined by the water cut engine 144 that processes data measured by the optoacoustic (infrared) transducer 116A. The GVF data 138 can be determined by the GVF engine 146 that processes data indicative of a mixture density measured by the acoustics receiver 116B.

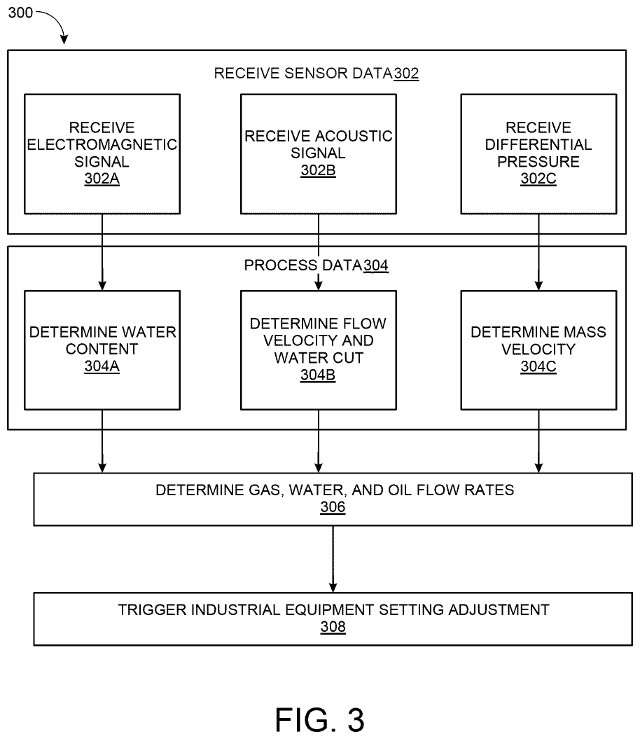

The flow rate engine 130 can include a data integration engine 148 and a prediction model 150 for characterizing the multiphase mixture 108 flowing through the pipe 110 in the industrial system 102. The data integration engine 148 can integrate the mass velocity data 134, the water cut data 136, and the gas volume fractions (GVF) data 138, to generate the flow rate data 140. The flow rate engine 130 can simulate the complex optoacoustic response of the multiphase fluid. The flow rate engine 130 can accommodate the full range of multiphase fractions (WC and GVF) with accuracy across wide range of process parameters, such as salinity and temperature. The volumetric flow rates of the different components of multiphase mixtures (including oil, water, and gas) are determined in two stages. Initially, the flow rate engine 130 can quantify the composition of the mixture 108, based on flow measurements received from the sensor 112. The flow rate engine 130 can determine the actual volumetric flow of the mixture, utilizing a volumetric phase fraction to mixture density conversion to determine two phase boundaries for each of the three phases included in the multiphase mixture, to characterize the composition of the multiphase mixture within a particular timeframe. The flow rate engine 130 can account for changes in conductivity, salinity, and temperature. The flow rate engine 130 can process data received from multiphase meters that exploit the difference of opto-acoustic properties of oil, water, and gas to distinguish different ratios in the mixture. Accounting for salinity and temperature, the flow rate engine 130 can accurately determine the composition of the multiphase mixture by using the overall mixture's volumetric flow rate along with the real-time flow rates of the individual components (oil, water, and gas). The composition of the multiphase mixture defines the volumetric fractions (as percentage) of each of oil water and gas at a particular time point. Further details about the operations of the flow rate engine 130 are described with reference to FIGS. 2A-2F and 3.

The prediction model 150 can leverage the results from all measurements recorded by the light source 114A, a light sensor 114B, one or more optical lenses 114C, an optoacoustic transducer 116A, an acoustics receiver 116B, and a differential pressure sensor 118A to mitigate systematic errors. For instance, GVF data 138 can rely on mixture density, which itself depends on water cut data 136 obtained from the infrared transducer 116A. The prediction model 150 can refine the correlation between the GVF data 138 and the water cut data 136, to improve the accuracy of flow rate data 140. The prediction model 150 can include a machine learning model trained to recognize a correlation pattern changes with respect to multiphase mixture composition, conductivity, and temperature. For example, the prediction model 150 can include support vector machines, neural networks, random forest, or a principal component analysis with machine learning model. Support vector machines are effective for classification tasks and can be used to identify flow pattern recognition. Neural networks, such as convolutional neural networks and recurrent neural networks, can capture intricate patterns in resonance frequency and quality factor trends available as time-series data. Random forest models can combine multiple decision trees to improve prediction accuracy, by handling large datasets and identifying important mixture features influencing resonance frequency patterns. The prediction model 150 can be trained on an extensive data set including measurements of mixtures with known phase distributions. The prediction model 150 can be trained on data that includes fluid conductivity, temperature, and optoacoustic and pressure measurements to identify the underlying patterns indicative of water cut data 136. The prediction model 150 can be validated on an extensive test data set including temperature, and optoacoustic and pressure measurements of mixtures with known phase distributions.

The action plan engine 132 can control operation of the sensor system 112 and initiate remediation operations based on the water cut data 136. For example, the action plan engine 132 can compare the determined composition of the multiphase mixture to a target composition of the multiphase mixture that can be generated by modifying settings of the flow controller 122. The action plan engine 132 can compare a determined profile of the multiphase mixture to a target profile of the multiphase mixture to generate action signals transmitted to the equipment controller 120 to modify operations of the industrial system 102.

The processor 126 can include a central processing unit, an application particular integrated circuit, a field-programmable gate array, or another suitable component. Generally, the processor 126 executes instructions and manipulates data for multiphase flow measurement. The processor 126 executes a functionality required to monitor multiphase mixture flow through different pipes of an industrial plant for anomaly detection and remediation.

In some implementations, the network 106 can include a large computer network, such as a local area network, a wide area network, the Internet, a cellular network, a telephone network, or an appropriate combination thereof connecting any number of communication devices, mobile computing devices, fixed computing devices and server systems. Data exchanged over the network 106, is transferred using any number of network layer protocols, such as internet protocol, multiprotocol label switching, asynchronous transfer mode, Frame Relay, etc. Furthermore, in implementations where the network 106 represents a combination of multiple sub-networks, different network layer protocols are used at each of the underlying sub-networks. In some implementations, the network 106 represents one or more interconnected internetworks, such as the public Internet.

FIG. 2A illustrates an example data collection system 200A that can be used to execute implementations of the present disclosure. FIG. 2B illustrates another example data collection system 200B that can be used to execute implementations of the present disclosure. FIG. 2C illustrates another example data collection system 200C that can be used to execute implementations of the present disclosure. FIG. 2D illustrates another example data collection system 200D that can be used to execute implementations of the present disclosure. FIG. 2E illustrates another example data collection system 200E that can be used to execute implementations of the present disclosure. FIG. 2F illustrates another example data collection system 200E that can be used to execute implementations of the present disclosure. FIGS. 2A, 2C, and 2E show cross sectional views. FIGS. 2B, 2D, and 2F show transversal sectional views of the example data collection systems shown in. FIGS. 2A, 2C, and 2E. FIGS. 2A-2F show different embodiments of the proposed solution in which the sensor system 112 presents a modularity feature to the sensor design that can be integrated within the inner volume of the pipe through a single slot 202 or a couple of adjacent slots 202A, 202B formed in the wall of the pipe 110. The slot gap can range from approximately 1 mm to approximately 3 mm.

The sensor system 112 can form a modular sensor that facilitates insertion through the gap in the pipe wall of its components that include the light source 114A, the light sensor 114B, the optical lenses 114C, the optoacoustic transducer 116A, the acoustics receiver 116B, and the differential pressure sensor 118A. The components of the sensor system 112 can be attached to or integrated in a housing 206 having a modular designed that it can hold all components securely. The housing 206 can have a resilient design against the flow to prevent any damage or displacement of any components of the sensor system 112. The housing 206 can have a shape that provides a minimal interference with the multiphase mixture flow (e.g., to prevent flow turbulence). For example, the housing 206 can have a conical, a cylindrical or baluster shape. The housing 206 can have a maximum diameter matching the diameter of the slot gap 202. At least a portion of the housing 206 can be formed from flexible materials to facilitate insertion and removal from the pipe 110. The flexible materials can facilitate a sealing insertion of the housing 206 within the slot 202 or slots 202A, 202B that prevents leakage of the multiphase mixture through the gap. The flexible materials can include any of rubber, nitrile, fluorocarbon, silicone, polyurethane, polytetrafluoroethylene, or any other material that can be highly resistant to chemicals and can operate at high temperatures.

The light source 114A can include a compact led and the light sensor 114B can include a laser diode or a miniaturized photodiode or phototransistor. The light sensor 114B can convert the detected reflected and refracted light into electromagnetic signals that can include a frequency shift relative to the light beam transmitted by the light source 114A. The light source 114A and the light sensor 114B are aligned to maximize light detection efficiency. The optical lenses 114C can include a small, high-quality optical lenses to focus or diffuse the light as needed. The optical lenses 114C can be securely mounted within the housing 206 to maintain optical alignment.

The optoacoustic transducer 116A and acoustics receiver 116B can include a miniaturized optoacoustic transducer that can convert light signals into acoustic waves. The optoacoustic transducer 116a can be paired with a sensitive acoustics receiver 116B to detect reflected and refracted acoustic waves that can include a frequency shift relative to the acoustics wave transmitted by the optoacoustic transducer 116A. The differential pressure sensor 118A can include a compact differential pressure sensor that can fit through the 1 mm gap 202.

FIG. 3 depicts a flowchart illustrating an example process 300 for multiphase flow measurement using microwave-based sensing, in accordance with some example implementations. Referring to FIGS. 1, 2A-2F, 4, and 5, the process 300 can be performed by any components of the example systems 100, 200A, 200B, 200C, 400, or 500. Operations of the process 300 are described below for illustration purposes only. Operations of the process 300 can be performed by any appropriate device or system, e.g., any appropriate data processing apparatus. Operations of the process 300 can also be implemented as instructions stored on a computer readable medium which can be non-transitory. Execution of the instructions causes one or more data processing apparatus to perform operations of the process 300.

At 302, sensor data are received from a sensor system attached to or integrated within a pipe in an industrial plant. The sensor data includes an electromagnetic signal, an acoustic signal, and a differential pressure. The sensor system includes a modular system at least partially insertable through a gap formed in a wall of the pipe. The sensor system includes a light source, a light sensor, one or more optical lenses, an optoacoustic transducer, an acoustics receiver, and a differential pressure sensor. The sensor system can generate measurements as a time series including at least two time points spaced at a set time interval (e.g., every 100 milliseconds or “ms”) reflecting change in the flow of the multiphase mixture over time. In some implementations, the sensor system includes a differential pressure transducer, a temperature transducer, and a pressure/temperature sensor box that can generate additional flow measurement data indicative of the flow of the multiphase mixture flowing through an industrial pipe. The sensor system can be installed in an industrial plant, such as a hydrocarbon production or processing plant that can include one or more operating wells and pipes facilitating the flow of the hydrocarbons from one system to another. The sensor system can be configured to activate data collection and/or transmission according to a respective schedule defining a frequency of data collection and a duration of each collection duration. The sensor system can be configured to collect data continuously (according to the respective schedule) or can have a set trigger that initiates data collection in response to detection of one or more conditions for data collection.

Receiving the sensor data includes, at 302A, receiving, by one or more processors of a server system, from a light receiver of an industrial system, an electromagnetic signal generated by an electromagnetic wave reflected by a multiphase mixture flowing through a pipe. Receiving the sensor data includes, at 302B, receiving, by the one or more processors from a transducer, an acoustic signal reflected by the multiphase mixture flowing through the pipe. Receiving the sensor data includes, at 302C, receiving, by the one or more processors from a differential pressure sensor, a differential pressure of the multiphase mixture flowing through the pipe.

At 304, the received sensor data is processed, by the one or more processors to generate processed data including mass velocity data, water cut data, and GVF data. In particular, processing the received sensor data includes, at 304A, determining, by the one or more processors, a water content of the multiphase mixture flowing through the pipe from the electromagnetic signal. The water content of the multiphase mixture flowing through the pipe can be calculated by considering that the absorption of the infrared light is mainly dependent on the water molecule. The water content calculation is independent of the salinity because water absorption is based on the water molecule itself, being independent from what is dissolved in the water. The water content calculation can include a calibration of each transducer, such that for infrared transducers two measurement one at 0% water-cut and another at 100% water. A calibration curve that correlates the infrared absorption to known water content levels. This involves measuring the infrared absorption for mixtures with known water contents and plotting the results. The water content C can be defined in relationship to the infrared absorption A and a calibration constant k as:

$$C = A / k$$

Processing the received sensor data includes, at 304B, determining, by the one or more processors, the mixture density of the fluid flowing through the pipe. The water-cut processed from 304A can be used with the measured Speed of sound to determine the mixture density, which consequently helps to determine GVF. A calibration of the optoacoustic transducer calibration is performed by taking three measurements at 100% Gas, 100% water, and 100% oil. Speed of sound is mainly used to determine the mixture density that is directly related the calculated travel time of the sound wave transmitted inside the slots. The time-based flow measurements facilitate the determination of water-fraction content in the presence of three-phases (oil, water, and gas), and further allows for the prediction of flow patterns of the fluid passing through the industrial pipe. The speed of sound in a mixture is a function of the densities and sound speeds of the individual phases, as well as their phase fractions.

Processing the received sensor data includes, at 304C, determining, by the one or more processors, a mass velocity of the multiphase mixture flowing through the pipe from the differential pressure of the multiphase mixture. The total mass velocity can be determined from the differential pressure drop using Bernoulli's principle defining the relationship between pressure, density, velocity, acceleration due to gravity, and height.

At 306, a composition of the multiphase mixture is estimated by determining gas, water, and oil volumetric flow rates based on correlating the water content, the mixture velocity, and the mass velocity of the multiphase mixture flowing through the pipe. The composition of the multiphase mixture defines the volumetric fractions (as percentage) of each of oil water and gas at a particular time point. The estimation of the composition of the multiphase mixture can include an analysis performed by a prediction model trained to leverage the results from sensor measurements to mitigate systematic errors based on the interrelationship between the processed data including mass velocity data, water cut data, and GVF data. For instance, GVF data can rely on mixture density, which itself depends on water cut data obtained from the infrared transducer. The prediction model can refine the correlation between the GVF data, and the water cut data, to improve the accuracy of flow rate data.

The prediction model can include a machine learning model trained to recognize a pattern of mass velocity data changes, GVF data changes, and water cut data changes with respect to multiphase mixture composition. The machine learning model can include a machine learning model pre-trained and fine-tuned to identify matching reservoir characterization. In some implementations, the machine learning model can be based on machine learning techniques related to a deep neural network (DNN). A deep neural network can be referred to as a network because it can be represented by connecting different functions. For example, a model of the DNN can be represented as a graph representing how the functions are connected from an input layer, through one or more hidden layers, and finally to an output layer, and each layer can have one or more nodes. In an example, the DNN of the subject technology generates a dynamic characterization of multiphase mixtures using the training data sets as templates, with low computational requirements. The DNN model can provide quantitative value for the match between of the recorded and simulated patterns using the labeled patterns of pattern of mass velocity data changes, GVF data changes, and water cut data changes with respect to multiphase mixture composition. In one or more implementations, relationships between the received pattern data and simulated data can be determined during training of the DNN. The training step optimizes the weights and biases in the hidden and output layer such that the estimation error between the estimated mixture compositions and observed mixture compositions can be minimized. Estimation error can be root mean square deviation, or a composite of root mean square deviation, cross-correlation, or a geoscience error metric. To avoid overfitting during training, regularization of the estimation error is performed based upon the norms of weights in the hidden layers that are added to the estimation error. An optimization process can include application of a stochastic gradient descent algorithm (or any other appropriate optimization algorithm), which can use one or more iterative optimization techniques and/or use a small subset of the training dataset or batch with training samples randomly selected at a time. The variances calculated based upon the horizontal and vertical semi-variograms are included in the input feature. The optimization process can optimize the weights and biases associated with the vertical and horizontal semi-variances, and other input features such that an error in the mixture composition estimates relative to the observed mixture composition can be minimized. The process of training described here not only can minimize the error in mixture composition estimates, but also can incorporate changes with respect to conductivity and temperature. Following the completion of training that can be determined by the estimation error on the validation dataset falling below a cut-off value, the testing dataset can be used to determine the performance of the trained DNN on unseen data records that were not previously used for training. Although a DNN was discussed for the purposes of explanation, it is appreciated that the machine learning model can include other trainable machine learning techniques. Further, it is appreciated that other types of neural networks can be utilized by the subject technology. For example, a convolutional neural network, regulatory feedback network, radial basis function network, recurrent neural network, modular neural network, instantaneously trained neural network, spiking neural network, regulatory feedback network, dynamic neural network, neuro-fuzzy network, compositional pattern-producing network, memory network, and/or any other appropriate type of neural network can be utilized.

At 308, an automatic adjustment of an equipment setting is automatically triggered based on the determined composition of the multiphase mixture. The adjustment of an equipment setting can include a control operation of the equipment to regulate a flow of the multiphase mixture. For example, the determined composition of the multiphase mixture can be compared to a target composition of the multiphase mixture and the comparison can be used to modify settings of the flow controller. For example, the adjustment of an equipment setting can include a modification of system component operations for adjusting pressure, temperature, and/or volume, for example by valve and/or pump control. The adjustment of an equipment setting can be transmitted to be displayed by a graphical user interface.

The example process 300 allows the usage of a resilient sensor for accurate characterization of a multiphase mixture flowing through a pipe. The example process 300 can be scheduled and automated, being initiated for continuous multiphase flow analysis. The example process 300 provides accurate and consistent assessment results, by accounting for interdependence between derived data, advantageously facilitating an accurate prediction of the multiphase mixture composition. The data generated during the example process 300 is displayed on a user-friendly interface including various dashboards and reports, enabling a comprehensive mixture composition analysis. The data generated during the example process 300 can automatically update equipment settings for multiphase fluid flow management.

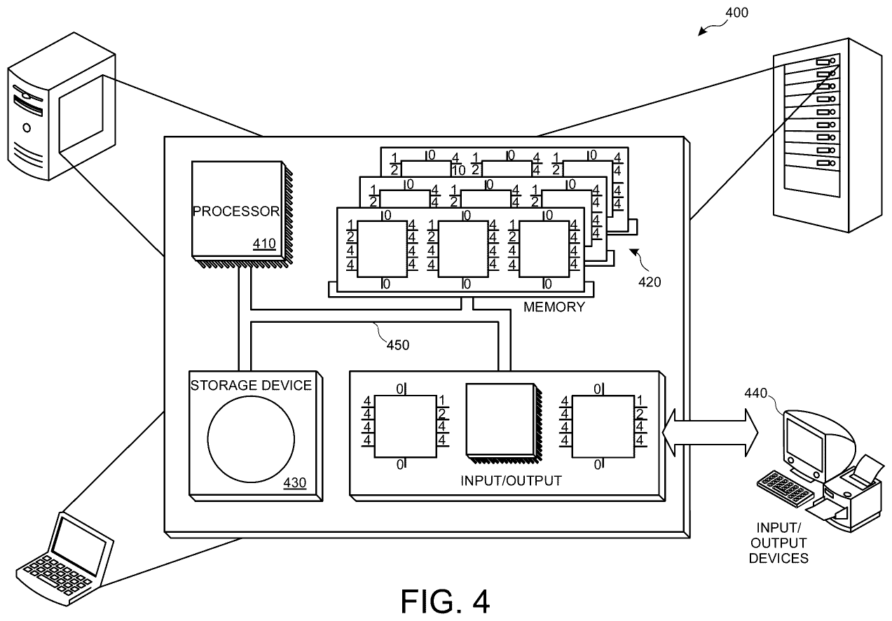

FIG. 4 depicts a block diagram illustrating a computing system 400, in accordance with some example implementations. Referring to FIG. 1, the computing system 400 can be used to implement the server system 104 and/or any other components of the example system 100.

As shown in FIG. 4, the computing system 400 can include a processor 410, a memory 420, a storage device 430, and input/output devices 440. The processor 410, the memory 420, the storage device 430, and the input/output devices 440 can be interconnected using a system bus 450. The processor 410 is capable of processing instructions for execution within the computing system 400. Such executed instructions can implement one or more components of, for example, the example system 100. In some implementations of the current subject matter, the processor 410 can be a single-threaded processor. Alternately, the processor 410 can be a multi-threaded processor. The processor 410 is capable of processing instructions stored in the memory 420 and/or on the storage device 430 to display graphical information for a user interface provided using the input/output device 440.

The memory 420 is a computer readable medium such as volatile or non-volatile that stores information within the computing system 400. The memory 420 can store data structures representing configuration object databases, for example. The storage device 430 is capable of providing persistent storage for the computing system 400. The storage device 430 can be a floppy disk device, a hard disk device, an optical disk device, or a tape device, or other suitable persistent storage means. The input/output device 440 provides input/output operations for the computing system 400. In some implementations of the current subject matter, the input/output device 440 includes a keyboard and/or pointing device. In various implementations, the input/output device 440 includes a display unit for displaying graphical user interfaces.

According to some implementations of the current subject matter, the input/output device 440 can provide input/output operations for a network device. For example, the input/output device 440 can include Ethernet ports or other networking ports to communicate with one or more wired and/or wireless networks (e.g., a local area network (LAN), a wide area network (WAN), the Internet).

In some implementations of the current subject matter, the computing system 400 can be used to execute various interactive computer software applications that can be used for organization, analysis and/or storage of data in various (e.g., tabular) format (e.g., Microsoft Excel®, and/or any other type of software). Alternatively, the computing system 400 can be used to execute any type of software applications. These applications can be used to perform various functionalities, e.g., planning functionalities (e.g., generating, managing, editing of spreadsheet documents, word processing documents, and/or any other objects), computing functionalities, or communications functionalities. The applications can include various add-in functionalities or can be standalone computing products and/or functionalities. Upon activation within the applications, the functionalities can be used to generate the user interface provided using the input/output device 440. The user interface can be generated and presented to a user by the computing system 400 (e.g., on a computer screen monitor).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random-access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

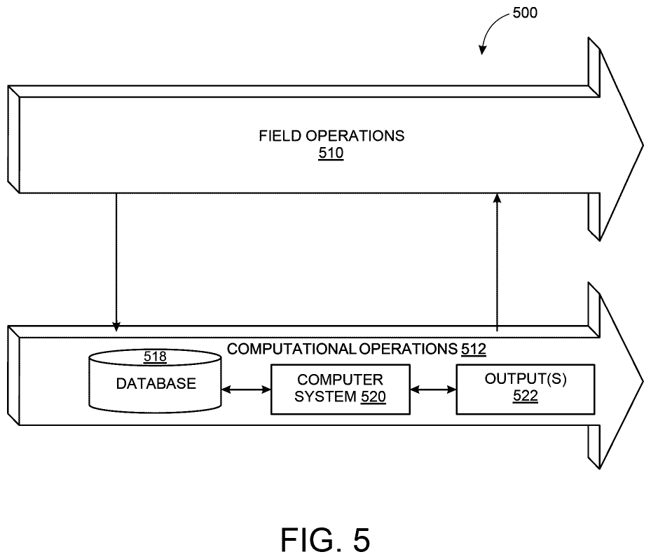

FIG. 5 illustrates hydrocarbon production operations 500 that include both one or more field operations 510 and one or more computational operations 512, which exchange information and control exploration for the production of hydrocarbons. In some implementations, outputs of techniques of the present disclosure can be performed before, during, or in combination with the hydrocarbon production operations 500, specifically, for example, either as field operations 510 or computational operations 512, or both.

Examples of field operations 510 include forming/drilling a wellbore, hydraulic fracturing, producing through the wellbore, injecting fluids (such as water) through the wellbore, to name a few. In some implementations, methods of the present disclosure can trigger or control the field operations 510. For example, the methods of the present disclosure can generate data from hardware/software including sensors and physical data gathering equipment (e.g., seismic sensors, well logging tools, flow meters, and temperature and pressure sensors). The methods of the present disclosure can include transmitting the data from the hardware/software to the field operations 510 and responsively triggering the field operations 510 including, for example, generating plans and signals that provide feedback to and control physical components of the field operations 510. Alternatively, or in addition, the field operations 510 can trigger the methods of the present disclosure. For example, implementing physical components (including, for example, hardware, such as sensors) deployed in the field operations 510 can generate plans and signals that can be provided as input or feedback (or both) to the methods of the present disclosure.

Examples of computational operations 512 include one or more computer systems 520 that include one or more processors and computer-readable media (e.g., non-transitory computer-readable media) operatively coupled to the one or more processors to execute computer operations to perform the methods of the present disclosure. The computational operations 512 can be implemented using one or more databases 518, which store data received from the field operations 510 and/or generated internally within the computational operations 512 (e.g., by implementing the methods of the present disclosure) or both. For example, the one or more computer systems 520 process inputs from the field operations 510 to assess conditions in the physical world, the outputs of which are stored in the databases 518. For example, seismic sensors of the field operations 510 can be used to perform a seismic survey to map subterranean features, such as facies and faults. In performing a seismic survey, seismic sources (e.g., seismic vibrators or explosions) generate seismic waves that propagate in the earth and seismic receivers (e.g., geophones) measure reflections generated as the seismic waves interact with boundaries between layers of a subsurface formation. The source and received signals are provided to the computational operations 512 where they are stored in the databases 518 and analyzed by the one or more computer systems 520.

In some implementations, one or more outputs 522 generated by the one or more computer systems 520 can be provided as feedback/input to the field operations 510 (either as direct input or stored in the databases 518). The field operations 510 can use the feedback/input to control physical components used to perform the field operations 510 in the real world.

For example, the computational operations 512 can process the seismic data to generate three-dimensional (3D) maps of the subsurface formation. The computational operations 512 can use these 3D maps to provide plans for locating and drilling exploratory wells. In some operations, the exploratory wells are drilled using logging-while-drilling (LWD) techniques which incorporate logging tools into the drill string. LWD techniques can enable the computational operations 512 to process new information about the formation and control the drilling to adjust to the observed conditions in real-time.

The one or more computer systems 520 can update the 3D maps of the subsurface formation as information from one exploration well is received and the computational operations 512 can adjust the location of the next exploration well based on the updated 3D maps. Similarly, the data received from production operations can be used by the computational operations 512 to control components of the production operations. For example, production well and pipe data can be analyzed to predict slugging in pipes leading to a refinery and the computational operations 512 can control machine operated valves upstream of the refinery to reduce the likelihood of plant disruptions that run the risk of taking the plant offline.

In some implementations of the computational operations 512, customized user interfaces can present intermediate or final results of the above-described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or app), or at a central processing facility.

The presented information can include feedback, such as changes in parameters or processing inputs, that the user can select to improve a production environment, such as in the exploration, production, and/or testing of petrochemical processes or facilities. For example, the feedback can include parameters that, when selected by the user, can cause a change to, or an improvement in, drilling parameters (including drill bit speed and direction) or overall production of a gas or oil well. The feedback, when implemented by the user, can improve the speed and accuracy of calculations, streamline processes, improve models, and solve problems related to efficiency, performance, safety, reliability, costs, downtime, and the need for human interaction.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 10 s. While the requested data need not be displayed (or initiated for display) instantaneously, it is displayed (or initiated for display) without any intentional delay, considering processing limitations of a described computing system and time required to, for example, gather, accurately measure, analyze, process, store, or transmit the data.

Events can include readings or measurements captured by downhole equipment such as sensors, pumps, bottom hole assemblies, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart or are located in different countries or other jurisdictions.

The preceding figures and accompanying description illustrate example processes and computer implementable techniques. The environments and systems described above (or their software or other components) can contemplate using, implementing, or executing any suitable technique for performing these and other tasks. It will be understood that these processes are for illustration purposes only and that the described or similar techniques can be performed at any appropriate time, including concurrently, individually, in parallel, and/or in combination. In addition, many of the operations in these processes can take place simultaneously, concurrently, in parallel, and/or in different orders than as shown. Moreover, processes can have additional operations, fewer operations, and/or different operations, so long as the methods remain appropriate.

In other words, although the disclosure has been described in terms of certain implementations and generally associated methods, alterations and permutations of these implementations, and methods will be apparent to those skilled in the art. Accordingly, the above description of example implementations does not define or constrain the disclosure. Other changes, substitutions, and alterations are also possible without departing from the spirit and scope of the disclosure.

A number of implementations of the present disclosure have been described. Nevertheless, it will be understood that various modifications can be made without departing from the spirit and scope of the present disclosure. Accordingly, other implementations are within the scope of the following claims.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application.
Example 1. A computer-implemented method comprising: receiving, by one or more processors from a light receiver, an electromagnetic signal generated by an electromagnetic wave reflected by a multiphase mixture flowing through a pipe; receiving, by the one or more processors from a transducer, an acoustic signal reflected by the multiphase mixture flowing through the pipe; receiving, by the one or more processors from a differential pressure sensor, a differential pressure of the multiphase mixture flowing through the pipe; determining, by the one or more processors, a water content of the multiphase mixture flowing through the pipe from the electromagnetic signal; determining, by the one or more processors, a flow velocity and a water-cut of the multiphase mixture flowing through the pipe from the acoustic signal; determining, by the one or more processors, a mass velocity of the multiphase mixture flowing through the pipe from the differential pressure of the multiphase mixture; estimating, by the one or more processors, a composition of the multiphase mixture by determining gas, water, and oil volumetric flow rates based on the water content, the flow velocity, and the mass velocity of the multiphase mixture flowing through the pipe; and triggering, by the one or more processors, an adjustment of a setting of an industrial equipment based on the composition of the multiphase mixture.
Example 2. The computer-implemented method of the previous example, wherein the light receiver is placed inside an opening of the pipe being exposed to the multiphase mixture flowing through the pipe.
Example 3. The computer-implemented method of any of the previous examples, wherein the light receiver comprises an infrared sensor.
Example 4. The computer-implemented method of any of the previous examples, wherein the transducer comprises an optoacoustic transducer and an acoustic receiver.
Example 5. The computer-implemented method of any of the previous examples, wherein the differential pressure sensor comprises a flow restrictor producing a pressure change in the multiphase mixture flowing through the pipe.
Example 6. The computer-implemented method of any of the previous examples, wherein determining, by the one or more processors, the water content comprises determining, by the one or more processors, a gas volume fraction from a mixture density.
Example 7. The computer-implemented method of any of the previous examples, further comprising:
   correlating the gas volume fraction to the water-cut of the multiphase mixture flowing through the pipe.
Example 8. The computer-implemented method of any of the previous examples, wherein the light receiver, the transducer and the differential pressure sensor are included in a housing, at least a portion of the housing being inserted within the pipe through a wall gap.
Example 9. A computer-implemented system comprising: memory storing application programming interface (API) information; and a server performing operations comprising: receiving, by one or more processors from a light receiver, an electromagnetic signal generated by an electromagnetic wave reflected by a multiphase mixture flowing through a pipe; receiving, by the one or more processors from a transducer, an acoustic signal reflected by the multiphase mixture flowing through the pipe; receiving, by the one or more processors from a differential pressure sensor, a differential pressure of the multiphase mixture flowing through the pipe; determining, by the one or more processors, a water content of the multiphase mixture flowing through the pipe from the electromagnetic signal; determining, by the one or more processors, a flow velocity and a water-cut of the multiphase mixture flowing through the pipe from the acoustic signal; determining, by the one or more processors, a mass velocity of the multiphase mixture flowing through the pipe from the differential pressure of the multiphase mixture; estimating, by the one or more processors, a composition of the multiphase mixture by determining gas, water, and oil volumetric flow rates based on the water content, the flow velocity, and the mass velocity of the multiphase mixture flowing through the pipe; and triggering, by the one or more processors, an adjustment of a setting of an industrial equipment based on the composition of the multiphase mixture.
Example 10. The computer-implemented system of the previous example, wherein the light receiver is placed inside an opening of the pipe being exposed to the multiphase mixture flowing through the pipe.
Example 11. The computer-implemented system of any of the previous examples, wherein the light receiver comprises an infrared sensor.
Example 12. The computer-implemented system of any of the previous examples, wherein the transducer comprises an optoacoustic transducer and an acoustic receiver.
Example 13. The computer-implemented system of any of the previous examples, wherein the differential pressure sensor comprises a flow restrictor producing a pressure change in the multiphase mixture flowing through the pipe.
Example 14. The computer-implemented system of any of the previous examples, wherein determining, by the one or more processors, the water content comprises determining, by the one or more processors, a gas volume fraction from a mixture density.
Example 15. The computer-implemented system of any of the previous examples, wherein the operations further comprise: correlating the gas volume fraction to the water-cut of the multiphase mixture flowing through the pipe.
Example 16. The computer-implemented system of any of the previous examples, wherein the light receiver, the transducer and the differential pressure sensor are included in a housing, at least a portion of the housing being inserted within the pipe through a wall gap.
Example 17. A non-transitory computer-readable media encoded with a computer program, the computer program comprising instructions that when executed by one or more computers cause the one or more computers to perform operations comprising: receiving, by one or more processors from a light receiver, an electromagnetic signal generated by an electromagnetic wave reflected by a multiphase mixture flowing through a pipe; receiving, by the one or more processors from a transducer, an acoustic signal reflected by the multiphase mixture flowing through the pipe; receiving, by the one or more processors from a differential pressure sensor, a differential pressure of the multiphase mixture flowing through the pipe; determining, by the one or more processors, a water content of the multiphase mixture flowing through the pipe from the electromagnetic signal; determining, by the one or more processors, a flow velocity and a water-cut of the multiphase mixture flowing through the pipe from the acoustic signal; determining, by the one or more processors, a mass velocity of the multiphase mixture flowing through the pipe from the differential pressure of the multiphase mixture; estimating, by the one or more processors, a composition of the multiphase mixture by determining gas, water, and oil volumetric flow rates based on the water content, the flow velocity, and the mass velocity of the multiphase mixture flowing through the pipe; and triggering, by the one or more processors, an adjustment of a setting of an industrial equipment based on the composition of the multiphase mixture.
Example 18. The non-transitory computer-readable media of the previous example, wherein the light receiver is placed inside an opening of the pipe being exposed to the multiphase mixture flowing through the pipe, and wherein the light receiver comprises an infrared sensor.
Example 19. The non-transitory computer-readable media of any of the previous examples, wherein the transducer comprises an optoacoustic transducer and an acoustic receiver, wherein the differential pressure sensor comprises a flow restrictor producing a pressure change in the multiphase mixture flowing through the pipe and wherein determining, by the one or more processors, the water content comprises determining, by the one or more processors, a gas volume fraction from a mixture density.
Example 20. The non-transitory computer-readable media of any of the previous examples, wherein the light receiver, the transducer and the differential pressure sensor are included in a housing, at least a portion of the housing being inserted within the pipe through a wall gap.

What is claimed is:

1. A computer-implemented method comprising:
receiving, by one or more processors from a light receiver, an electromagnetic signal generated by an electromagnetic wave reflected by a multiphase mixture flowing through a pipe;
receiving, by the one or more processors from a transducer, an acoustic signal reflected by the multiphase mixture flowing through the pipe;
receiving, by the one or more processors from a differential pressure sensor, a differential pressure of the multiphase mixture flowing through the pipe;
determining, by the one or more processors, a water content of the multiphase mixture flowing through the pipe from the electromagnetic signal;
determining, by the one or more processors, a flow velocity and a water-cut of the multiphase mixture flowing through the pipe from the acoustic signal;
determining, by the one or more processors, a mass velocity of the multiphase mixture flowing through the pipe from the differential pressure of the multiphase mixture;
estimating, by the one or more processors, a composition of the multiphase mixture by determining gas, water, and oil volumetric flow rates based on the water content, the flow velocity, and the mass velocity of the multiphase mixture flowing through the pipe; and
triggering, by the one or more processors, an adjustment of a setting of an industrial equipment based on the composition of the multiphase mixture.

2. The computer-implemented method of claim 1, wherein the light receiver is placed inside an opening of the pipe being exposed to the multiphase mixture flowing through the pipe.

3. The computer-implemented method of claim 1, wherein the light receiver comprises an infrared sensor.

4. The computer-implemented method of claim 1, wherein the transducer comprises an optoacoustic transducer and an acoustic receiver.

5. The computer-implemented method of claim 1, wherein the differential pressure sensor comprises a flow restrictor producing a pressure change in the multiphase mixture flowing through the pipe.

6. The computer-implemented method of claim 3, wherein determining, by the one or more processors, the water content comprises determining, by the one or more processors, a gas volume fraction from a mixture density.

7. The computer-implemented method of claim 6, further comprising:
correlating the gas volume fraction to the water-cut of the multiphase mixture flowing through the pipe.

8. The computer-implemented method of claim 1, wherein the light receiver, the transducer and the differential pressure sensor are included in a housing, at least a portion of the housing being inserted within the pipe through a wall gap.

9. A computer-implemented system comprising:
memory storing application programming interface (API) information; and
a server performing operations comprising:
receiving, by one or more processors from a light receiver, an electromagnetic signal generated by an electromagnetic wave reflected by a multiphase mixture flowing through a pipe;
receiving, by the one or more processors from a transducer, an acoustic signal reflected by the multiphase mixture flowing through the pipe;
receiving, by the one or more processors from a differential pressure sensor, a differential pressure of the multiphase mixture flowing through the pipe;
determining, by the one or more processors, a water content of the multiphase mixture flowing through the pipe from the electromagnetic signal;
determining, by the one or more processors, a flow velocity and a water-cut of the multiphase mixture flowing through the pipe from the acoustic signal;
determining, by the one or more processors, a mass velocity of the multiphase mixture flowing through the pipe from the differential pressure of the multiphase mixture;
estimating, by the one or more processors, a composition of the multiphase mixture by determining gas, water, and oil volumetric flow rates based on the water content, the flow velocity, and the mass velocity of the multiphase mixture flowing through the pipe; and
triggering, by the one or more processors, an adjustment of a setting of an industrial equipment based on the composition of the multiphase mixture.

10. The computer-implemented system of claim 9, wherein the light receiver is placed inside an opening of the pipe being exposed to the multiphase mixture flowing through the pipe.

11. The computer-implemented system of claim 9, wherein the light receiver comprises an infrared sensor.

12. The computer-implemented system of claim 9, wherein the transducer comprises an optoacoustic transducer and an acoustic receiver.

13. The computer-implemented system of claim 9, wherein the differential pressure sensor comprises a flow restrictor producing a pressure change in the multiphase mixture flowing through the pipe.

14. The computer-implemented system of claim 11, wherein determining, by the one or more processors, the water content comprises determining, by the one or more processors, a gas volume fraction from a mixture density.

15. The computer-implemented system of claim 14, wherein the operations further comprise:
correlating the gas volume fraction to the water-cut of the multiphase mixture flowing through the pipe.

16. The computer-implemented system of claim 9, wherein the light receiver, the transducer and the differential pressure sensor are included in a housing, at least a portion of the housing being inserted within the pipe through a wall gap.

17. A non-transitory computer-readable media encoded with a computer program, the computer program comprising instructions that when executed by one or more computers cause the one or more computers to perform operations comprising:
receiving, by one or more processors from a light receiver, an electromagnetic signal generated by an electromagnetic wave reflected by a multiphase mixture flowing through a pipe;
receiving, by the one or more processors from a transducer, an acoustic signal reflected by the multiphase mixture flowing through the pipe;
receiving, by the one or more processors from a differential pressure sensor, a differential pressure of the multiphase mixture flowing through the pipe;
determining, by the one or more processors, a water content of the multiphase mixture flowing through the pipe from the electromagnetic signal;
determining, by the one or more processors, a flow velocity and a water-cut of the multiphase mixture flowing through the pipe from the acoustic signal;
determining, by the one or more processors, a mass velocity of the multiphase mixture flowing through the pipe from the differential pressure of the multiphase mixture;
estimating, by the one or more processors, a composition of the multiphase mixture by determining gas, water, and oil volumetric flow rates based on the water content, the flow velocity, and the mass velocity of the multiphase mixture flowing through the pipe; and
triggering, by the one or more processors, an adjustment of a setting of an industrial equipment based on the composition of the multiphase mixture.

18. The non-transitory computer-readable media of claim 17, wherein the light receiver is placed inside an opening of the pipe being exposed to the multiphase mixture flowing through the pipe, and wherein the light receiver comprises an infrared sensor.

19. The non-transitory computer-readable media of claim 17, wherein the transducer comprises an optoacoustic transducer and an acoustic receiver, wherein the differential pressure sensor comprises a flow restrictor producing a pressure change in the multiphase mixture flowing through the pipe and wherein determining, by the one or more processors, the water content comprises determining, by the one or more processors, a gas volume fraction from a mixture density.

20. The non-transitory computer-readable media of claim 17, wherein the light receiver, the transducer and the differential pressure sensor are included in a housing, at least a portion of the housing being inserted within the pipe through a wall gap.
